(19) United States
(12) Patent Application Publication
(10) Pub. No.: US 2016/0143532 A1
(43) Pub. Date: May 26, 2016

(54) WEARABLE DEVICE AND METHOD OF CONTROLLING THE SAME

(71) Applicant: Samsung Electronics Co., Ltd., Suwon-si (KR)

(72) Inventors: Sang-Young LEE, Anyang-si (KR); Dae-Hyun KIM, Hwaseong-si (KR)

(21) Appl. No.: 14/947,433

(22) Filed: Nov. 20, 2015

(30) Foreign Application Priority Data
Nov. 21, 2014 (KR) 10-2014-0163730

(51) Int. Cl. A61B 5/00

(52) U.S. Cl. CPC A61B 5/0024

(57) ABSTRACT

A wearable device and a control method thereof are provided. The wearable device includes a processor, a sensor module configured to obtain biometric information about a user wearing the wearable device, and a light emitting module configured to output visual information corresponding to the obtained biometric information, wherein the processor controls an output attribute of the visual information to be changed and output according to a change in the obtained biometric information.

### CROSS-REFERENCE TO RELATED APPLICATION(S)

This application claims the benefit under 35 U.S.C. §119(a) of a Korean patent application filed on Nov. 21, 2014 in the Korean Intellectual Property Office and assigned Serial number 10-2014-0163730, the entire disclosure of which is hereby incorporated by reference.

### TECHNICAL FIELD

The present disclosure relates to a wearable device and a method of controlling the same.

### BACKGROUND

Recently, electronic devices, such as a smart phone, have rapidly penetrated the consumer market, so that most people have at least one such device now. This means that the electronic device has become an important part of the user's daily life, and in actuality, the user/users recognize that it would be difficult to return to a life without the electronic device after they have come to rely on it.

Similar to the rapid supply of the electronic device, a supply of a wearable device, which performs various functions while being connected with the electronic device, has been increased. Examples of the wearable device include various devices, such as a smart watch (e.g., Galaxy Gear™ of Samsung, and the like) and a smart band. The wearable device may provide a user wearing the wearable device with various information through a display provided in the wearable device. The smart band is generally used while being worn on a wrist of a user, and may be provided to a user/users in a form of a narrow bracelet or bangle.

The above information is presented as background information only to assist with an understanding of the present disclosure. No determination has been made, and no assertion is made, as to whether any of the above might be applicable as prior art with regard to the present disclosure.

### SUMMARY

However, according to the smart band in the related art, the smart band is generally provided to the user/users in the form of a bracelet as described above, so that a display mode provided in the smart band inevitably has a relatively small size. Accordingly, the user of the smart band obtains information (for example, biometric information, such as a pulse rate, of the user, and information on a walking or running distance of the user) provided by the smart band through the small display module, so that it is difficult to accurately obtain information. Particularly, when the user does various activities (for example, jogging), it is more difficult to accurately check information provided through the small display module.

Aspects of the present disclosure are to address the above-mentioned problems and/or disadvantages and to provide at least the advantages described below. Accordingly, an aspect of the present disclosure is to provide a wearable device which is capable of intuitively providing a user with various pieces of information.

Another aspect of the present disclosure is to provide a method of controlling a wearable device which is capable of intuitively providing a user with various pieces of information.

Although technical merits of the present disclosure can be achieved by the mentioned aspect, other aspects of the present disclosure can be readily understood by those skilled in the art through the following description.

In accordance with an aspect of the present disclosure, a wearable device is provided. The wearable device includes a processor, a sensor module configured to obtain biometric information about a user wearing the wearable device, and a light emitting module configured to output visual information corresponding to the obtained biometric information, wherein the processor controls an output attribute of the visual information to be changed and output according to a change in the obtained biometric information.

In accordance with another aspect of the present disclosure a method of controlling a wearable device is provided. The method includes obtaining biometric information about a user wearing the wearable device, outputting visual information corresponding to the obtained biometric information, and changing an output attribute of the visual information according to a change of the obtained biometric information and outputting the visual information.

In accordance with another aspect of the present disclosure a wearable device is provided. The wearable device includes a light emitting module, a sensor module configured to detect a movement of a user wearing the wearable device, a communication module configured to receive information on a first distance set by the user, and a processor configured to calculate a ratio of the received first distance to a second distance determined based on a movement of the user, wherein the processor controls visual information corresponding to the calculated ratio to be output by the light emitting module.

According to the present disclosure, a user may intuitively recognize various pieces of information provided by the wearable device, so that even though the user may be engaged in various activities (for example, jogging), the user may accurately recognize the various pieces of information.

Further, according to the present disclosure, a user may intuitively recognize various pieces of information provided through the wearable device, so that it is possible to decrease a time taken for checking information provided through the wearable device compared to the related art, thereby reducing the risk of an unexpected accident occurring due to the user checking the information.

Other aspects, advantages, and salient features of the disclosure will become apparent to those skilled in the art from the following detailed description, which taken in conjunction with the annexed drawings, discloses various embodiments of the present disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram for describing a state in which a user wears a wearable device according to various embodiments of the present disclosure;

FIGS. 2A, 2B, and 2C are diagrams for describing a wearable device according to various embodiments of the present disclosure;

FIGS. 3A and 3B are diagrams for describing a wearable device according to various embodiments of the present disclosure;

FIGS. 4A, 4B, and 4C are diagrams for describing a function/operation of setting various operation modes of a wearable device through an input module of the wearable device according to various embodiments of the present disclosure;

FIGS. 5A, 5B, 5C, 5D, 5E, 5F, and 5G are diagrams for describing a function/operation of setting an environment for performing a first operation mode of a wearable device according to various embodiments of the present disclosure;

FIGS. 6A, 6B, 6C, 6D, 6E, 7A, 7B, 7C, and 8 are diagrams for describing various functions/operations performed in a first operation mode of a wearable device according to various embodiments of the present disclosure;

FIGS. 9A, 9B, 9C, 9D, 9E, and 9F are diagrams for describing a function/operation of changing a color displayed on the wearable device through an input module provided in a wearable device in a first operation mode according to various embodiments of the present disclosure;

FIG. 10 is a flowchart for describing a method of controlling a wearable device according to various embodiments of the present disclosure;

FIGS. 11A, 11B, 11C, 11D, 11E, 11F, 11G, 11H, 11I, and 11J are diagrams for describing various functions/operations performed in a second operation mode of a wearable device according to various embodiments of the present disclosure;

FIGS. 12A, 12B, and 12C are diagrams for describing a function/operation of changing a color displayed in a wearable device in the second operation mode of the wearable device according to various embodiments of the present disclosure;

FIG. 13 is a diagram for describing a function/operation simultaneously performed in various operation modes of a wearable device according to various embodiments of the present disclosure;

FIG. 14 is a flowchart for describing a method of controlling a wearable device according to various embodiments of the present disclosure; and

FIGS. 15A, 15B, and 15C are diagrams for describing various functions/operations performed in a third operation mode of the wearable device according to various embodiments of the present disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the present disclosure various as defined by the claims and their equivalents. It includes specific details to assist in that understanding but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Although the terms including an ordinal number such as first, second, etc. can be used for describing various elements, the structural elements are not restricted by the terms. The terms are used merely for the purpose to distinguish an element from the other elements. For example, a first element could be termed a second element, and similarly, a second element could be also termed a first element without departing from the scope of the present disclosure. As used herein, the term “and/or” includes any and all combinations of one or more associated items.

The terms used in this application is for the purpose of describing particular embodiments only and is not intended to limit the disclosure. As used herein, the singular forms are intended to include the plural forms as well, unless the context clearly indicates otherwise. In the description, it should be understood that the terms “include” or “have” indicate existence of a feature, a number, an operation, a structural element, parts, or a combination thereof, and do not previously exclude the existences or probability of addition of one or more another features, numeral, operations, structural elements, parts, or combinations thereof.

Unless defined differently, all terms used herein, which include technical terminologies or scientific terminologies, have the same meaning as that understood by a person skilled in the art to which the present disclosure belongs. It should be interpreted that the terms, which are identical to those defined in general dictionaries, have the meaning identical to that in the context of the related technique. The terms should not be ideally or excessively interpreted as a formal meaning.

A wearable device according to various embodiments of the present disclosure may include a wearable device having a shape of, for example, a “smart band” or an “electronic bracelet”. The smart band may be generally formed in a bracelet shape, so that a user may wear the smart band on a wrist or an ankle. However, in the present specification, for convenience of the description of the present disclosure, a wearable device having a shape of a smart band or an electronic bracelet has been described as an example, but various embodiments of the present disclosure are not limited by the example. The wearable device according to various embodiments of the present disclosure may include various devices, for example, smart glasses, a head mounted device (HMD), electronic clothes, an electronic necklace, an electronic appcessory, an electronic tattoo, a smart mirror, and a smart watch.

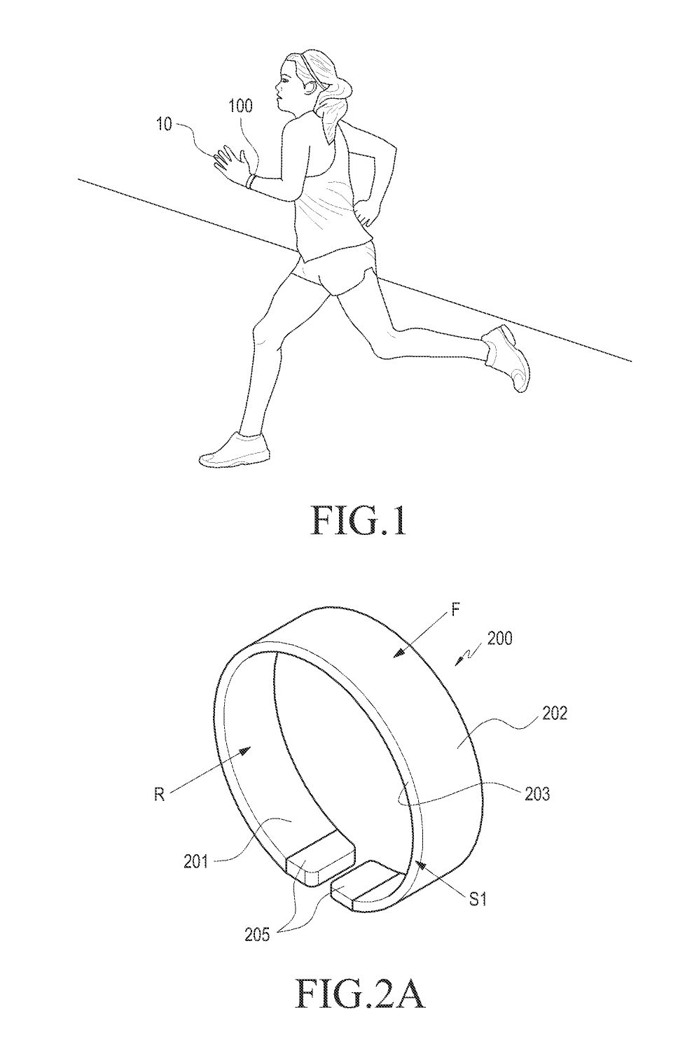

FIG. 1 is a diagram for describing a state in which a user wears a wearable device according to various embodiments of the present disclosure.

Referring to FIG. 1, a wearable device 100 according to various embodiments of the present disclosure may be worn on a wrist of a user 10. Otherwise, although not illustrated in FIG. 1, the wearable device 100 may also be worn on another part (for example, an ankle) of a body of the user 10. The wearable device 100 according to various embodiments of the present disclosure may be worn on the wrist or another part of the user 10 to provide the user 10 with various pieces of information, such as biometric information about the user, according to an activity (for example, an exercise including jogging) of the user 10.

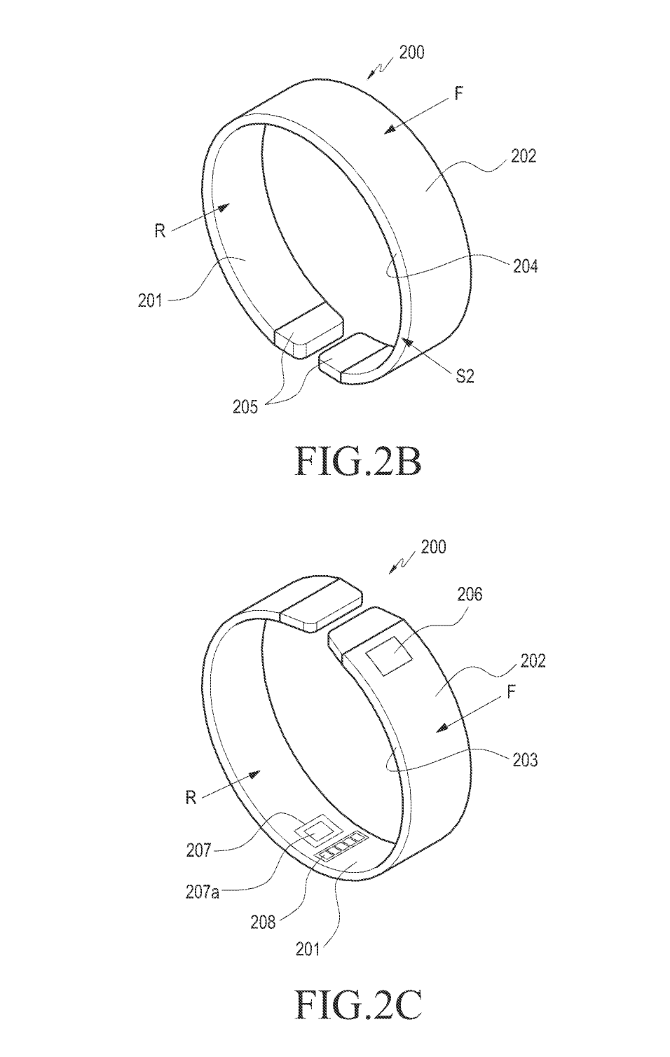

FIGS. 2A to 2C are diagrams for describing the wearable device according to various embodiments of the present disclosure.

Referring to FIGS. 2A and 2B, a wearable device 200 according to various embodiments of the present disclosure may have a shape having a predetermined curvature and being wearable on a body part, for example, a wrist or an ankle, of a user. The wearable device 200 may include a housing 201, a first light emitting module 202, a second light emitting module 203, a third light emitting module 204, and supporting parts 205.

The housing 201 may include a front surface F, a rear surface R, and side surfaces S1 and S2 connecting the front surface F and the rear surface R. Each of the front surface F and the rear surface R may be formed to have a predetermined curvature. The housing 201 may be made of a plastic material having elasticity so as to be easily detachable from a part of the body of the user. However, according to various embodiments of the present disclosure, the housing 201 may be made of various materials, such as glass or a ceramic material. The housing 201 may include a protection layer formed of a sheet material, such as polyethylene terephthalate (PET), for protecting a surface of the housing 201.

The first light emitting module 202 may be mounted in an internal space of the front surface F of the housing 201, and the front surface F of the housing 201 may be manufactured so as to cover the first light emitting module 202 mounted in the internal space of the front surface F. The user may recognize a color indicated by light output by the first light emitting module 202 while wearing the wearable device 200 on a part of the body of the user. To this end, the front surface F of the housing 201 may be formed of a transparent material or a semi-transparent material so that light output by the first light emitting module 202 may pass therethrough. The first light emitting module 202 may be manufactured based on a flexible printed circuit board, and mounted in the internal space of the front surface F of the housing 201 so as to accord with a curved design of the wearable device 200. The first light emitting module 202 may be set to output various colors.

The second light emitting module 203 and the third light emitting module 204 may be disposed at inner spaces of both side surfaces S1 and S2 of the housing 201, respectively, and the side surfaces S1 and S2 of the housing 201 may be manufactured so as to cover the second light emitting module 203 and the third light emitting module 204 mounted in the internal spaces of both side surfaces S1 and S2. The user may recognize a color indicated by light emitted by the first light emitting module 202 in a state where the wearable device 200 is worn on a part of the body of the user. To this end, both side surfaces S1 and S2 of the housing 201 may be formed of a transparent material or a semi-transparent material so that light output by the second light emitting module 203 and/or the third light emitting module 204 passes therethrough. The second light emitting module 203 and the third light emitting module 204 may be manufactured based on a flexible printed circuit board, and mounted in the inner spaces of the side surfaces S1 and S2 of the housing 201 so as to accord with a curved design of the wearable device 200.

The rear surface R of the housing 201 may be in contact with a part of the body of the user in the state where the wearable device 200 is worn on the part of the body of the user. A light emitting module may not be mounted in the internal space of the rear surface R of the housing 201. Accordingly, the rear surface R of the housing 201 may also be manufactured of an opaque material, as well as a transparent material or a semi-transparent material. A sensor unit 207 and a charging terminal unit 208 may be disposed on the rear surface R of the housing 201, which will be described below.

The support units 205 may provide predetermined frictional force between a part of the body of the user and the wearable device 200 in order to prevent the wearable device 200 from being separated from the part of the body of the user in the state where the wearable device 200 is worn on the part of the body of the user. To this end, the support unit 205 may be formed of a rubber or a silicon material.

Referring to FIG. 2C, the sensor unit 207 and the charging terminal unit 208 may be disposed on the rear surface R of the wearable device 200 according to various embodiments of the present disclosure. A button 206, for example, an on-off button, may be included. A sensor module 207a (for example, a biometric sensor) may be disposed in the sensor unit 207. The sensor module 207a may be mounted in the internal space of the rear surface R of the wearable device 200. For example, when the sensor module is a biometric sensor, the sensor module may irradiate light to a blood vessel of the user by using a light emitting diode, an infrared ray light emitting diode, and the like, to detect light reflected by using a light receiving element (for example, a photo diode), and sense a pulse rate, a blood flow rate, oxygen saturation and the like of the user. The sensor unit 207 may be manufactured in a depressed structure in which the sensor module 207a may be embedded. The depressed portion of the sensor unit 207 may be covered by a cover part made of a transparent plastic material. The charging terminal unit 208 may be electrically connected to a battery (not illustrated) embedded in the rear surface R of the housing 201 of the wearable device 200. A charging function for the battery may be provided to the user through the charging terminal unit 208.

A shape of the wearable device 200 illustrated in FIGS. 2A to 2C may be variously modified and manufactured in a manufacturing process of the wearable device 200. The wearable device 200 according to various embodiments of the present disclosure may be manufactured so as to have a different curvature from a curvature of the wearable device 200 illustrated in FIGS. 2A to 2C, and the wearable device 200 may be modified and manufactured so as to have various thicknesses and/or widths. The round wearable device 200 is exemplarily illustrated in FIGS. 2A to 2C, but the wearable device 200 according to various embodiments of the present disclosure may be manufactured so as to have various forms, such as a quadrangular form having a smooth edge.

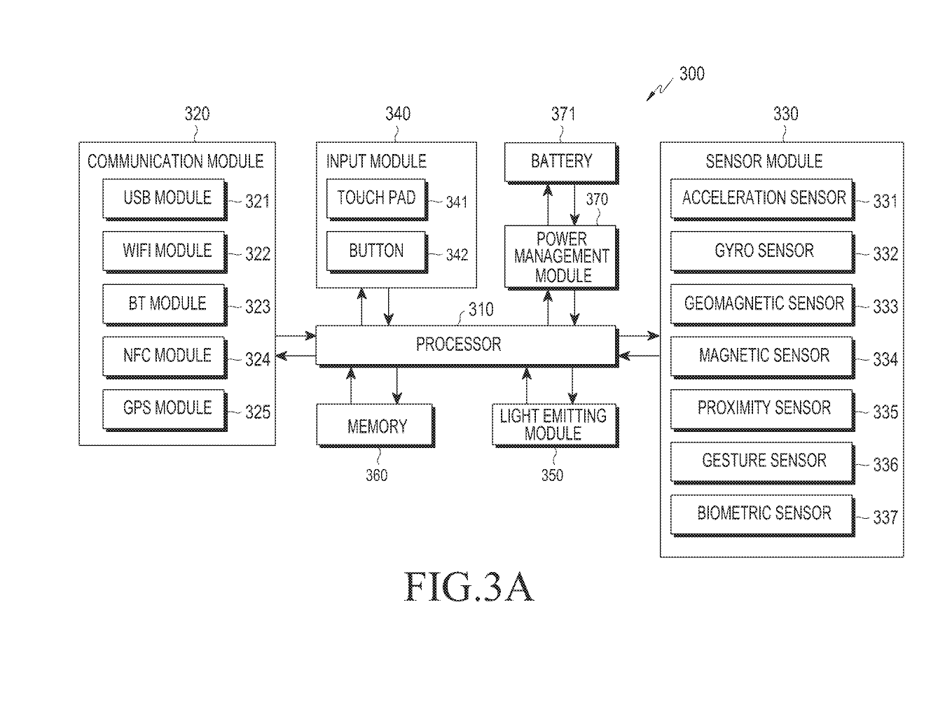

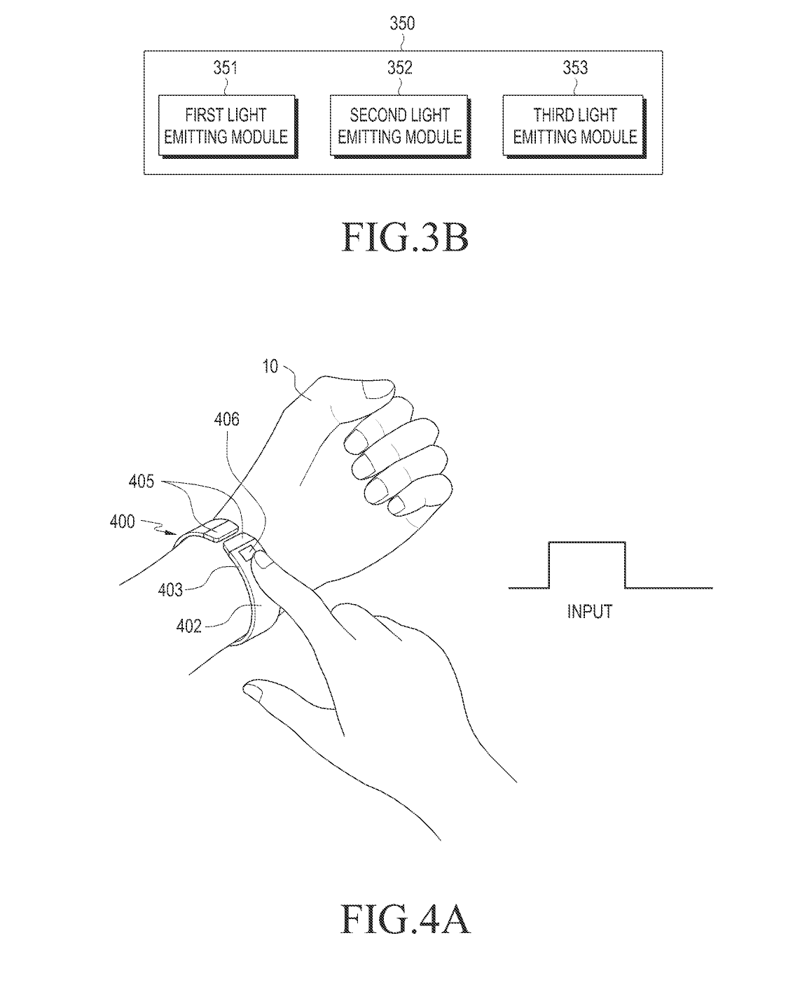

FIGS. 3A and 3B are block diagrams illustrating a wearable device according to various embodiments of the present disclosure.

Referring to FIG. 3A, a wearable device 300 according to various embodiments of the present disclosure may include a processor 310, a communication module 320, a sensor module 330, an input module 340, a light emitting module 350, and a power management module 370.

The processor 310 may include one or more of a central processing unit (CPU), an application processor (AP), a communication processor (CP), and a micro controller unit (MCU). The processor 310 may carry out operations or data processing related to control and/or communication of at least one other element of the electronic device 300.

The communication module 320 may transceiver data between the wearable device 300 and other external electronic devices (for example, a smart phone) connected through wired/wireless communication. According to various embodiments of the present disclosure, the communication module 320 may include a universal serial bus (USB) module 321, a WiFi module 322, a Bluetooth™ (BT) module 323, a near field communication (NFC) module 324, and a global positioning system (GPS) module 325. According to various embodiments of the present disclosure, at least three of the USB module 321, the WiFi module 322, the BT module 323, the NFC module 324, and the GPS module 325 may be included in one integrated circuit (IC) package.

The sensor module 330 may measure a physical quantity or detect an operation state of the wearable device 300, and convert the measured or detected information into an electric signal. The sensor module 330 according to various embodiments of the present disclosure may include, for example, an acceleration sensor 331, a gyro sensor 332, a geomagnetic sensor 333, a magnetic sensor 334, a proximity sensor 335, a gesture sensor 336, and a biometric sensor 337. Additionally or alternatively, the sensor module 330 may include a biometric recognizing sensor, for example, an olfactory sensor (E-nose sensor), an electromyography (EMG) sensor, an electroencephalogram (EEG) sensor, an electrocardiogram (ECG) sensor, an iris sensor, and a fingerprint sensor, and recognize biometric information about the user by using the biometric recognizing sensor. The sensor module 330 may further include a control circuit for controlling one or more sensors included therein.

The input module 340 may include a touch pad 341 and/or a button 342. The touch pad 341 may recognize a touch input of the user in at least one type among, for example, a capacitive type, a resistive type, an infrared type, and an ultrasonic type. The touch pad 341 may also further include a control circuit. The capacitive touch panel may recognize physical contact or proximity. The touch pad 341 may further include a tactile layer. In this case, the touch panel 341 may provide a tactile reaction to a user. The button 342 may include, for example, a physical button, an optical key, or a keypad. However, according to various embodiments of the present disclosure, the input module 340 may be omitted and manufactured in the manufacturing process of the wearable device 300.

The light emitting module 350 may include a first light emitting module 351, a second light emitting module 352, and a third light emitting module 353 as illustrated in FIG. 3B. The light emitting module 350 may include, for example, a light emitting diode. However, in addition to the light emitting diode, various light emitting elements may be applicable. Contents described with reference to FIGS. 2A to 2C are equally applicable to descriptions of the first light emitting module 351, the second light emitting module 352, and the third light emitting module 353.

The memory 360 may include a volatile memory and/or a non-volatile memory. The memory 360 may store, for example, commands or data related to one or more other components of the wearable device 300. According to various embodiments of the present disclosure, the memory 360 may store software and/or various programs.

The power management module 370 may manage power of the wearable device 300. Although not illustrated, the power managing module 370 may include, for example, a power management IC (PMIC), a charger IC (IC), or a battery fuel gauge. The PMIC may be mounted, for example, in integrated circuits or system on chip (SoC) semiconductors. The charging methods may be classified into wired charging and wireless charging. The charger IC may charge a battery and may prevent an overvoltage or excess current from being induced or flowing from a charger. According to an embodiment, the charger IC may include a charger IC for at least one of the wired charging method and the wireless charging method. A magnetic resonance scheme, a magnetic induction scheme, or an electromagnetic scheme may be exemplified as the wireless charging method, and an additional circuit for wireless charging, such as a coil loop circuit, a resonance circuit, a rectifier circuit, and the like may be added. The battery gauge may measure, for example, a residual quantity of the battery 371, and a voltage, a current, or a temperature during the charging. The battery 371 may store electricity and supply power. The battery 371 may include, for example, a rechargeable battery or a solar battery.

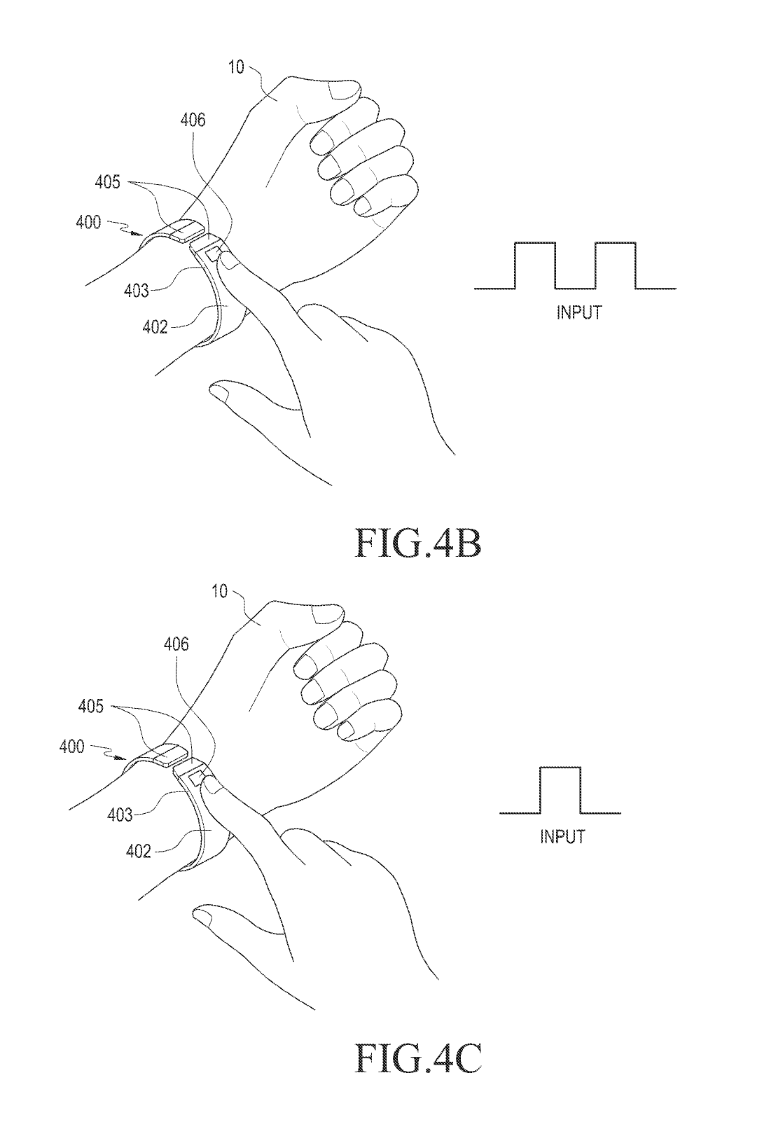

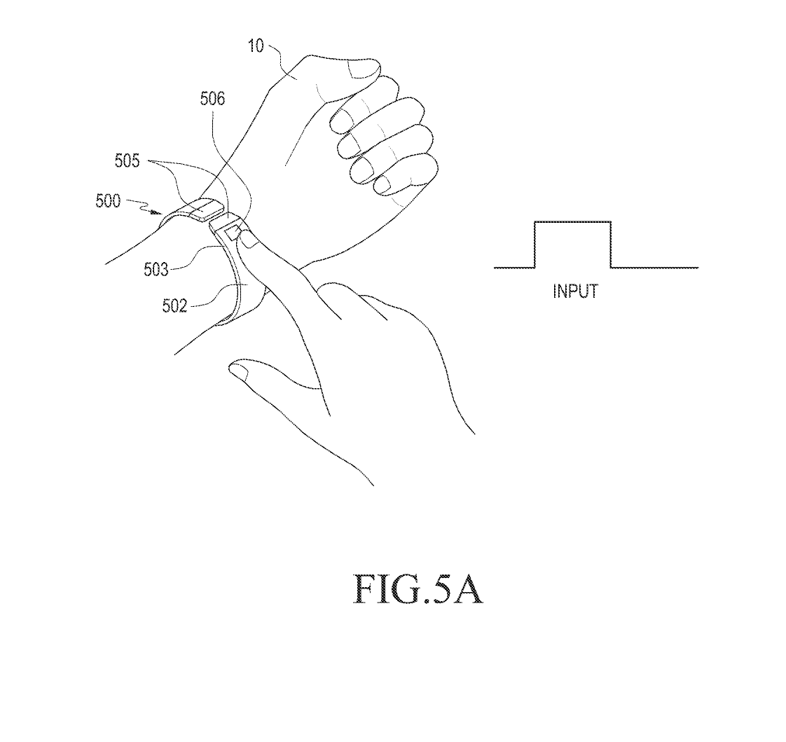

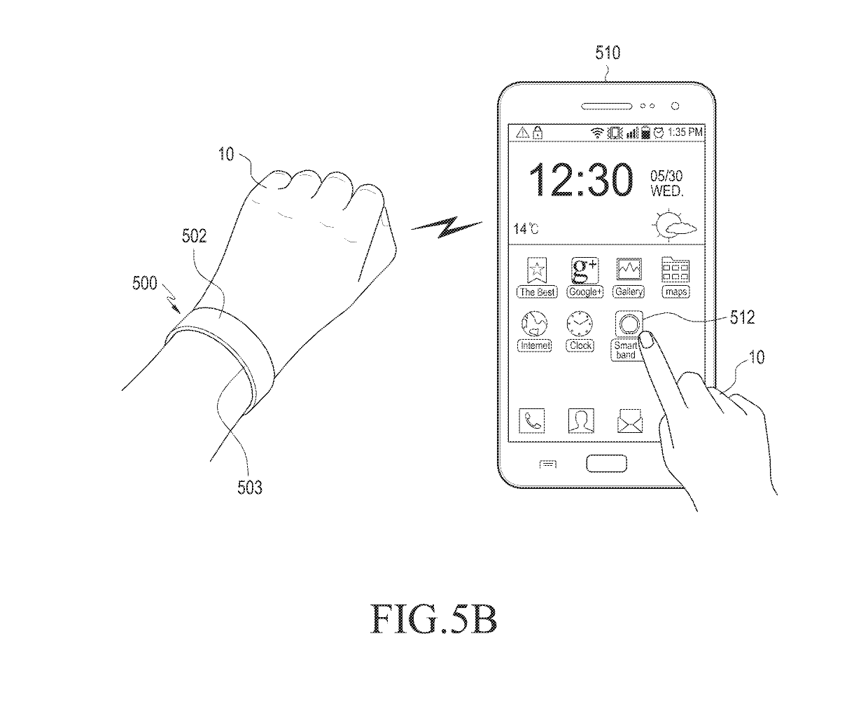

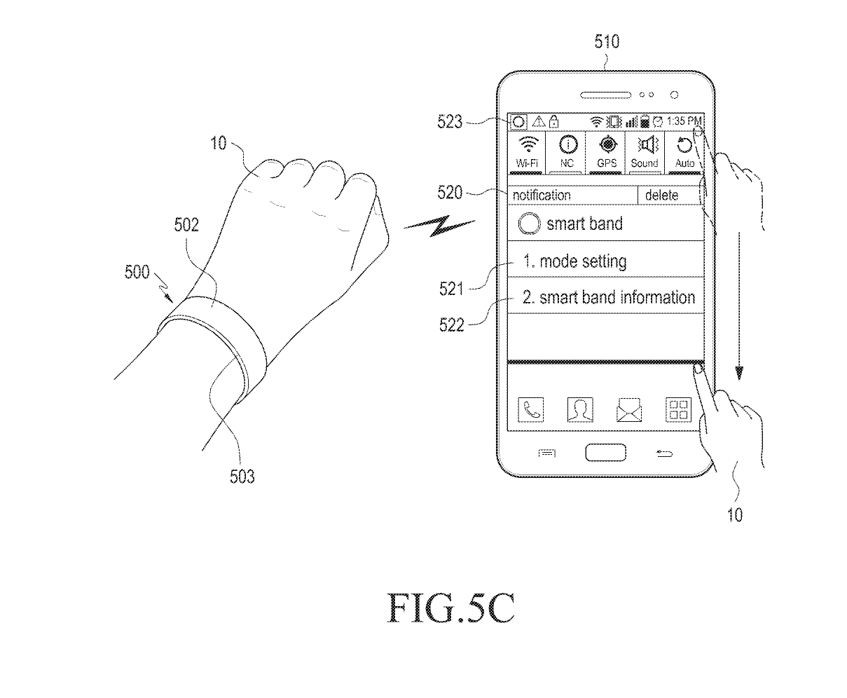

FIGS. 4A to 4C are diagrams for describing a function/operation of setting various operation modes of a wearable device through an input module of the wearable device according to various embodiments of the present disclosure. In FIGS. 4A to 4C, embodiments are illustrated, in which a wearable device 400 is not connected to an external electronic device (for example, a smart phone), but is dependently functioned/operated according to a manipulation of the user 10.

Referring to FIG. 4A, FIG. 4A illustrates a state where the user 10 wears the wearable device 400 according to various embodiments of the present disclosure. The wearable device 400 may include a first light emitting module 402, a second light emitting module 403, and supporting parts 405. The wearable device 400 may receive a turn-on/turn-off input from the user 10. The turn-on/turn-off input may be, for example, an input (for example, one long press) through a button 406. As illustrated in FIG. 4A, when a user input for the button 406 is received for a predetermined time or longer, a processor (for example, the processor 310) may control the wearable device 400 to be turned on/off. In FIG. 4A, the button 406 is illustrated as one embodiment of the input module, but the aforementioned touch pad (for example, the touch pad 341) may also be applied. When the wearable device 400 is turned on according to the input of the user 10, the processor may control an operation mode of the wearable device 400 to be set as a first operation mode. The first operation mode may also be referred to as a “healthcare mode” in the present specification. The first operation mode will be described in more detail below.

Referring to FIG. 4A, FIG. 4B illustrates an operation of receiving an input for switching the operation mode to a second operation mode (or a third operation mode), which is different from the first operation mode, in a state where the wearable device 400 is in the operated state in the first operation mode (or an operated state in the second operation mode). For example, when an input (for example, two short presses) of the button 406 is received from the user 10 as illustrated in FIG. 4B, the processor may control the operation mode of the wearable device 400 to be switched to the second operation mode (or the third operation mode). The second operation mode may also be referred to as an “activity mode” in the present specification, and the third operation mode may also be referred to as an “accessory mode” in the present specification. The second operation mode or the third operation mode will be described in more detail below.

Referring to FIG. 4C, when an input (for example, one short press) of the button 406 is received from the user during the execution of any one operation mode among the first to third operation modes as illustrated in FIG. 4C, the processor may execute a mode for changing a color displayed on the wearable device 400. This will be described in detail below.

The press types (that is, the method of pressing the button 406, such as the case of two short presses) for the button 406 mentioned in FIGS. 4A to 4C are illustrative for describing the present disclosure. The press type may be designated by various methods, and the embodiments about the press type are not limited by the aforementioned contents.

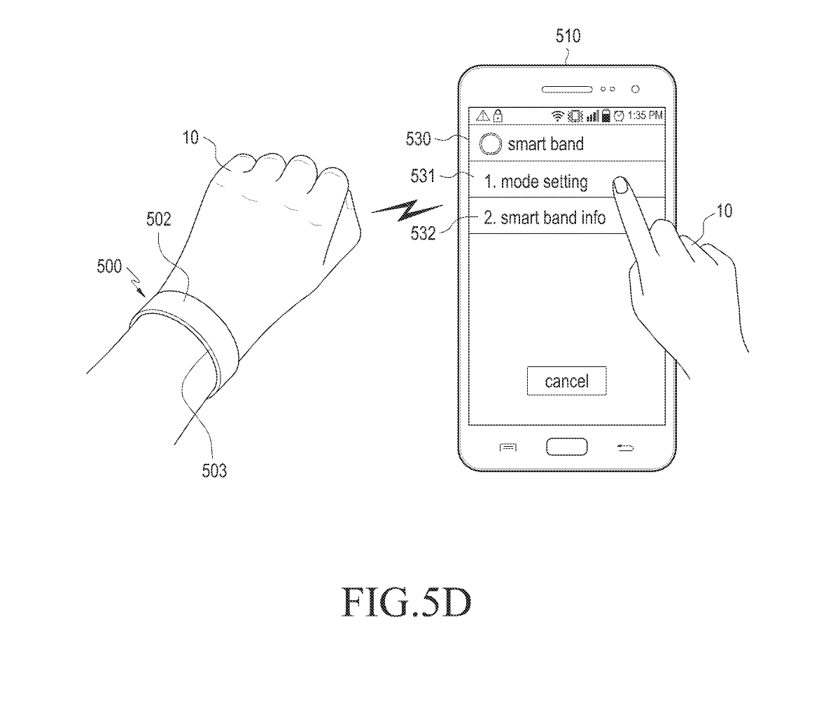

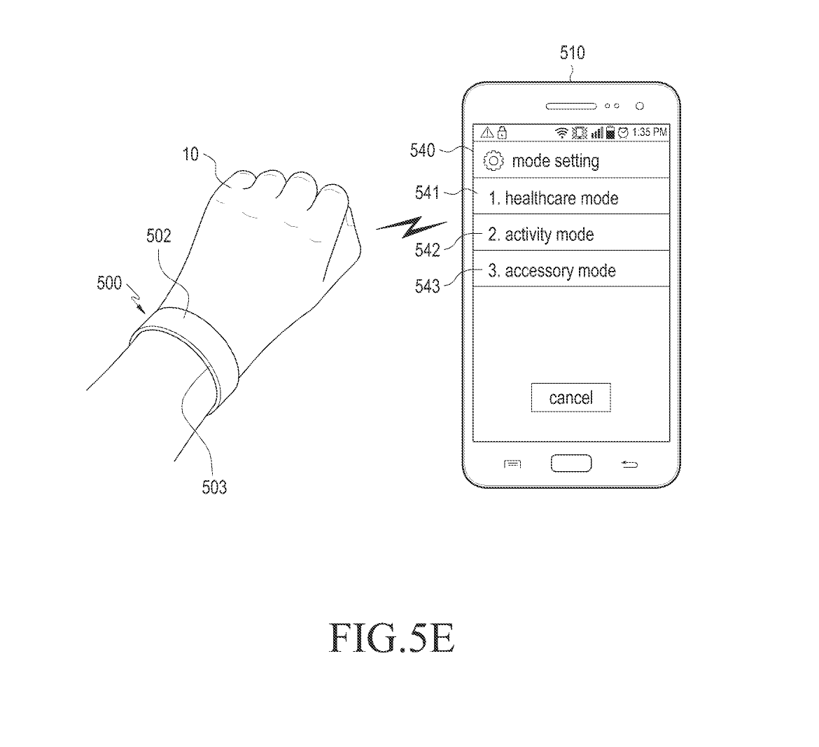

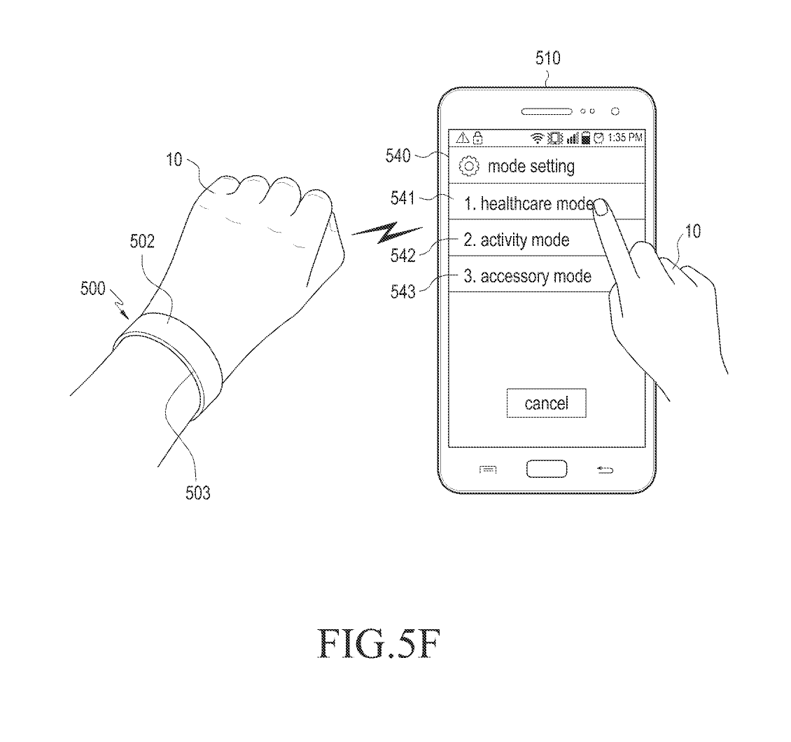

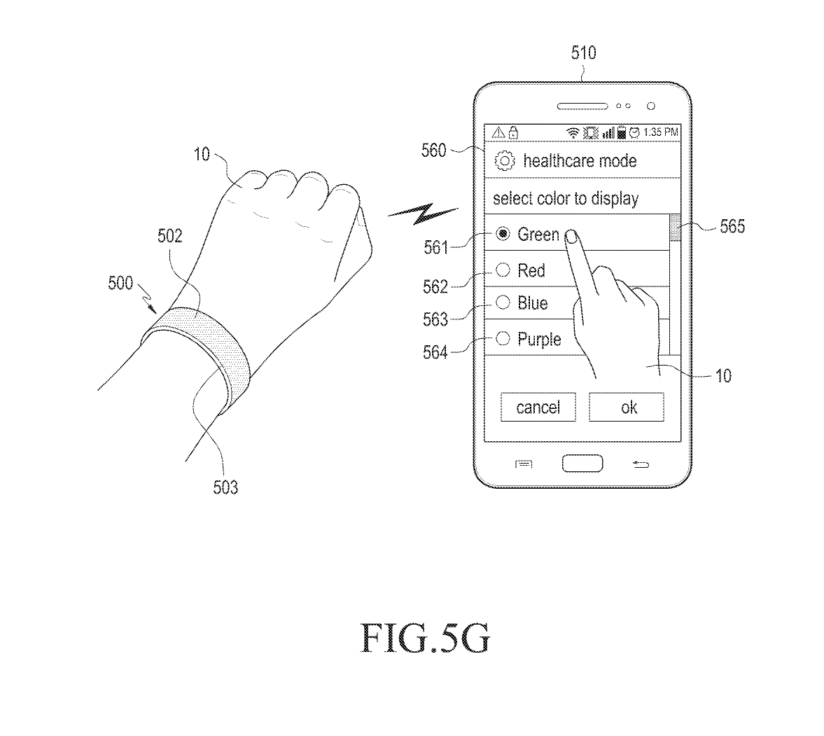

FIGS. 5A to 5G are diagrams for describing a function/operation of setting an environment for performing a first operation mode of a wearable device according to various embodiment of the present disclosure. FIGS. 5A to 5G illustrate embodiments in which a wearable device 500 according to various embodiments of the present disclosure is connected with an external electronic device (for example, a smart phone 510), so that the wearable device is controlled through the smart phone 510.

Referring to FIGS. 5A to 5C, various embodiments for the connection with the smart phone 510 are illustrated.

Referring to FIG. 5A, the wearable device 500 may include a first light emitting module 502, a second light emitting module 503, and supporting parts 505. When a processor (for example, the processor 310) receives an input (for example, one long press) for a button 506 of the wearable device 500, the processor may control a request message requesting for the connection with the smart phone 510 to be transmitted to the smart phone 510 positioned in the vicinity of the wearable device 500 through a communication module (for example, the communication module 320). According to the request message, when the smart phone 510 is normally connected with the wearable device 500, a screen 530 for setting an operation of the wearable device 500 may be displayed on the smart phone 510 as illustrated in FIG. 5D.

Referring to FIG. 5B, the smart phone 510 may be connected with the wearable device 500 positioned in the vicinity of the smart phone 510 by a selection of an application 512 installed in the smart phone 510. When the application 512 is selected, the smart phone 510 may search for the wearable device 500 positioned in the vicinity of the smart phone 510, and when the wearable device 500 is discovered, the smart phone 510 may be connected with the wearable device 500, and display the screen 530 for setting an operation mode of the wearable device 500 on the smart phone 510 as illustrated in FIG. 5D.

Referring to FIG. 5C, the smart phone 510 may search for the wearable device 500 positioned around the smart phone 510 according to a predetermined time period. When the wearable device 500 is discovered in the vicinity of the smart phone 510, the smart phone 510 may be connected with the wearable device 500, and display a notification window 520 for setting an operation mode of the wearable device 500. When the wearable device 500 is connected with the smart phone 510, the smart phone 510 may display a device icon 523. The user 10 may select items 521 and 522 displayed on the notification window 520, and select an operation mode of the wearable device 500 or check information about the wearable device 500.

Referring to FIGS. 5D to 5G, operations for setting an environment for executing the first operation mode are illustrated. The user 10 may select items 531 and 532 displayed on the notification window 530, and select an operation mode of the wearable device 500 or check information about the wearable device 500. As illustrated in FIG. 5D, when an item 531 for setting an operation mode of the wearable device 500 is selected by the user 10, the smart phone 510 may display a notification window 540 and various items 541, 542, and 543 for setting an operation mode of the wearable device 500 as shown in FIG. 5E. The healthcare mode (or the first operation mode 541) may mean a mode of obtaining, by the wearable device 500, biometric information (for example, a pulse cycle or a pulse rate) of the user wearing the wearable device 500 and outputting visual information corresponding to the obtained biometric information. The biometric information about the user 10 may be obtained through, for example, a sensor module (for example, the biometric sensor 337), and the visual information may include colors output by, for example, the first to third light emitting modules 202, 203, and 204. As illustrated in FIG. 5F, when the healthcare mode item 541 is selected by the user 10, the selection information may be transmitted to the wearable device 500. When the healthcare mode item 541 is selected, the smart phone 510 may display a color selection screen 560 for a color output by a first light emitting module 502. For example, the color selection screen 560 may include items for various colors green 561, red 562, blue 563, and purple 564. Additional colors may be available and brought into view by operation of a scroll bar 565. FIG. 5G exemplarily illustrates an operation of selecting green as the color. According to various embodiments of the present disclosure, as illustrated in FIG. 5G, all of the first to third light emitting modules 502, 503, and 504 may be controlled to output the color. The first light emitting module 502 may be controlled to output the selected color or a pre-designated color in the first operation mode. Through this, when there is a change in the biometric information, for example, when a sharp change of a pulse rate is detected, the wearable device 500 may indirectly notify neighboring people of the user 10, as well as the user 10, of a problem of a body of the user 10, so that the wearable device 500 may be used as a notification means for notifying of an emergency in an emergency situation.

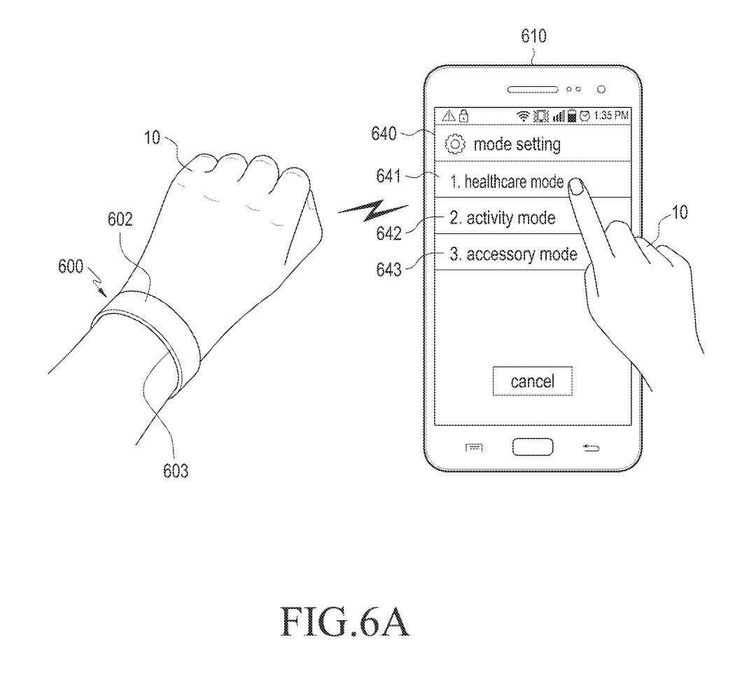

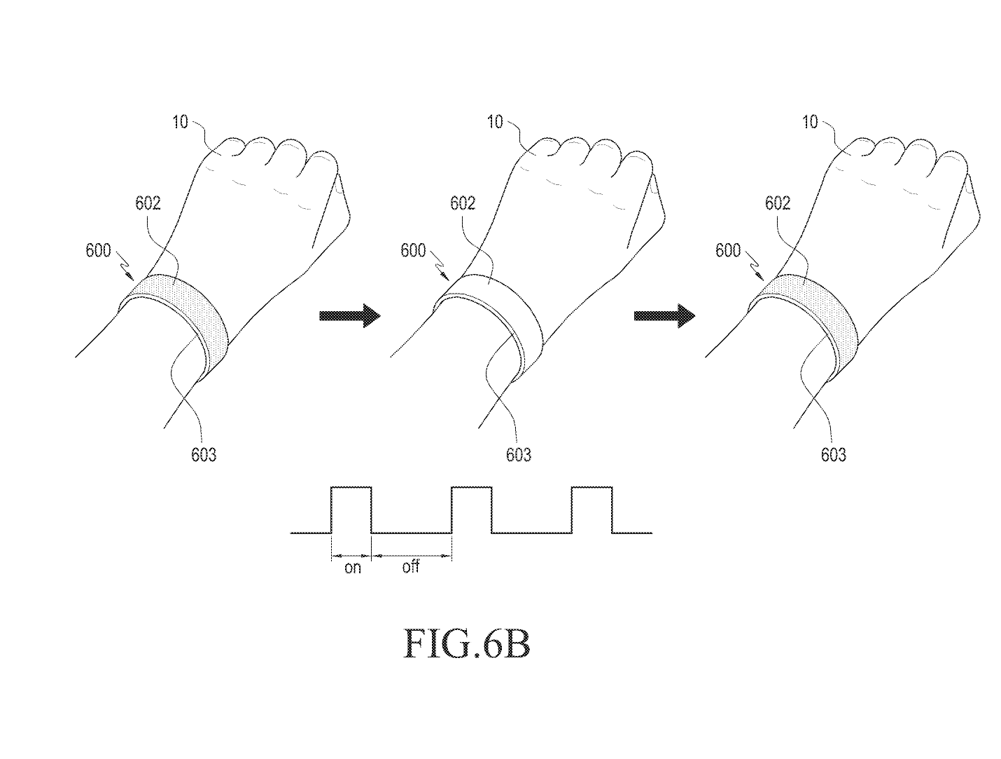

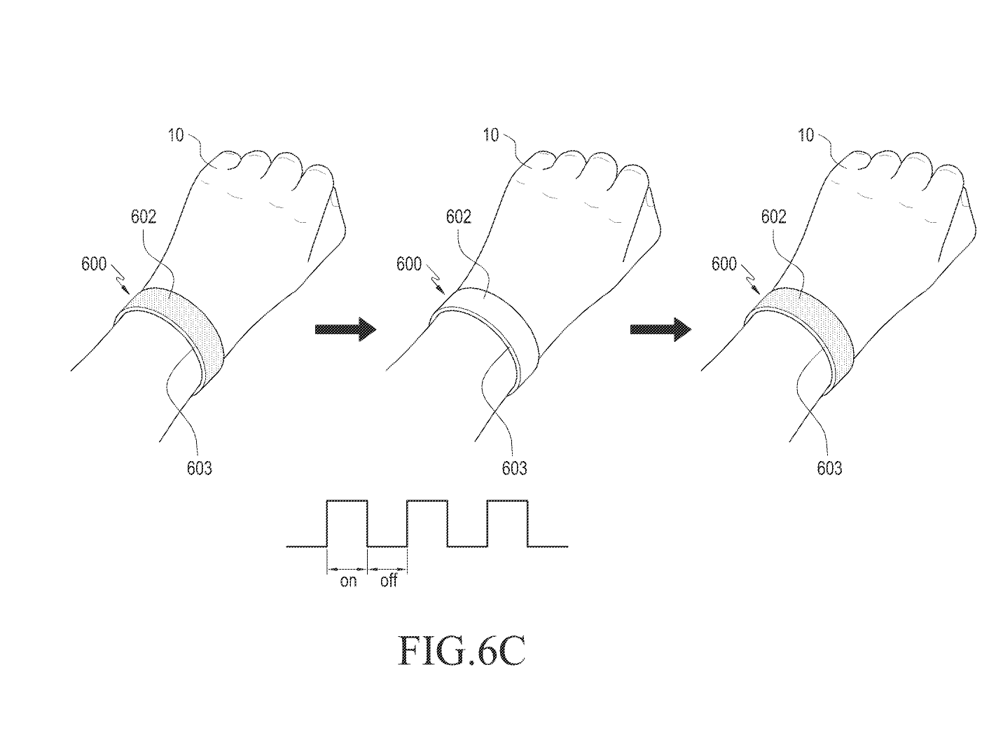

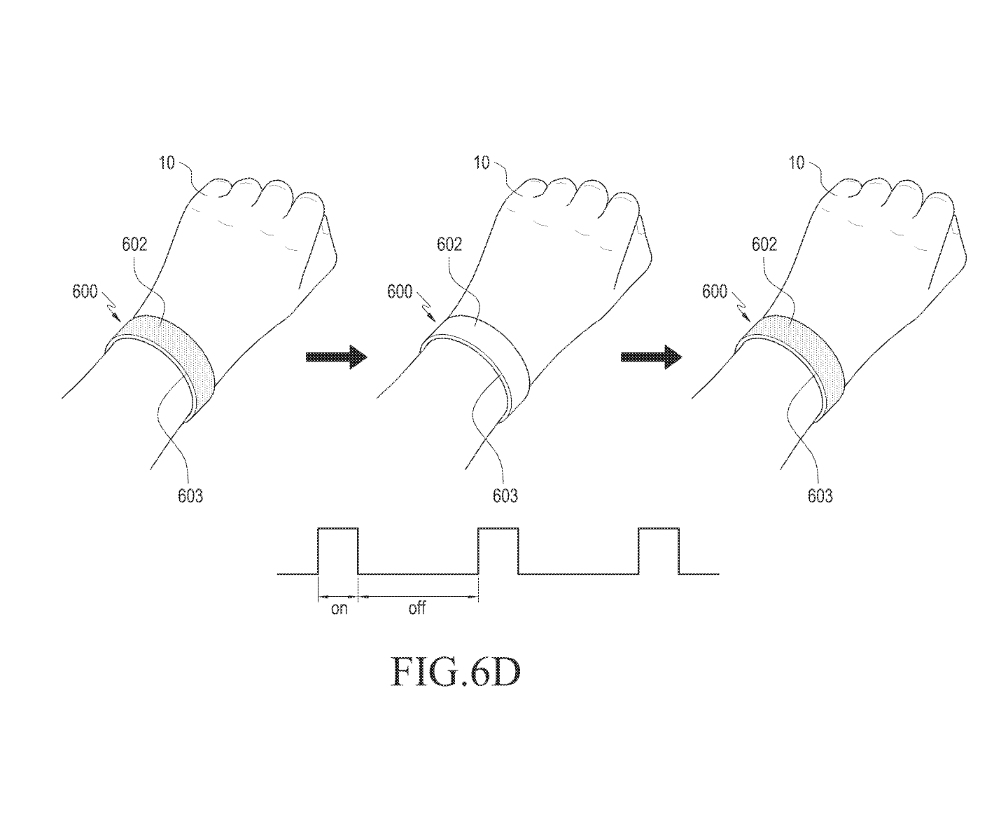

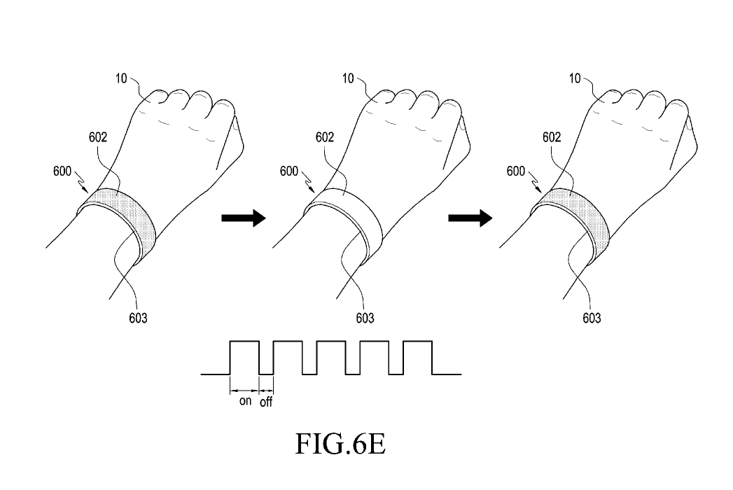

FIGS. 6A to 8 are diagrams for describing various functions/operations performed in a first operation mode of a wearable device according to various embodiments of the present disclosure.

Referring to FIG. 6A, when an item 531 for setting an operation mode of the wearable device 600 is selected by the user 10, the smart phone 610 may display a notification window 640 and various items 641, 642, and 643 for setting an operation mode of the wearable device 600. As described above, when a selection input for a healthcare mode item 641 by the user 10 is received in a smart phone 610, a wearable device 600 may be operated in the healthcare mode (i.e., a first operation mode).

At a time point at which an operation of the first operation mode starts, the wearable device 600 may obtain biometric information, for example, a pulse rate/pulse cycle, of the user 10 through a sensor module (for example, the biometric sensor 337). In the present specification, a phrase “obtain a pulse rate/pulse cycle” may be referred to as “detect a pulse rate/pulse cycle”.

A processor (for example, the processor 310) of the wearable device 600 may control an output attribute (for example, an output period) of the first light emitting module 602 or the second light emitting module 603 as illustrated in FIG. 6B in correspondence to the obtained pulse rate/pulse cycle of the user 10. A meaning of a phrase “the processor controls the output cycle of the first light emitting module 602 in correspondence to the pulse rage/pulse cycle” may include a meaning that the processor controls the output cycle so as to “correspond to the obtained pulse rate/pulse cycle”, or the processor controls the output cycle of the first light emitting module 602 according to “a predetermined output interval in correspondence to the pulse rate/pulse cycle” even though the output cycle does not correspond to the pulse rate/pulse cycle. Through the aforementioned function/functions or operation/operations, the user 10 may intuitively recognize his/her current pulse rate/pulse cycle through the output cycle.

FIG. 6C illustrates a case where a pulse rate/pulse cycle of the user 10 is increased compared to that of a case illustrated in FIG. 6B, and FIG. 6D illustrates a case where a pulse rate/pulse cycle of the user 10 is decreased compared to that of the case illustrated in FIG. 6B. Referring to FIGS. 6C and 6D, the processor may control an output period of the first light emitting module 602 to be changed and output in correspondence to the changed pulse rate/pulse cycle of the user 10.

Referring to FIG. 6E, according to various embodiments of the present disclosure, when a change in the pulse rate/pulse cycle of the user 10 is detected, the processor may not change the output cycle so as to correspond to the change of the pulse rate/pulse cycle, but may control the first light emitting module 602 to output a selected or predetermined color according to a predetermined output period. That is, when the “change” in the pulse rate/pulse cycle is simply detected based on the pulse rate/pulse cycle obtained at a specific time point (for example, a start time point of the operation of the first operation mode (FIG. 6A)), the processor may control the output period of the first light emitting module 602 according to a predetermined output period as illustrated in FIG. 6E.

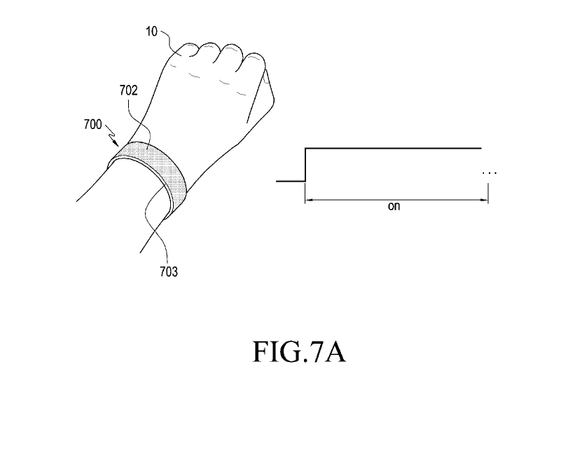

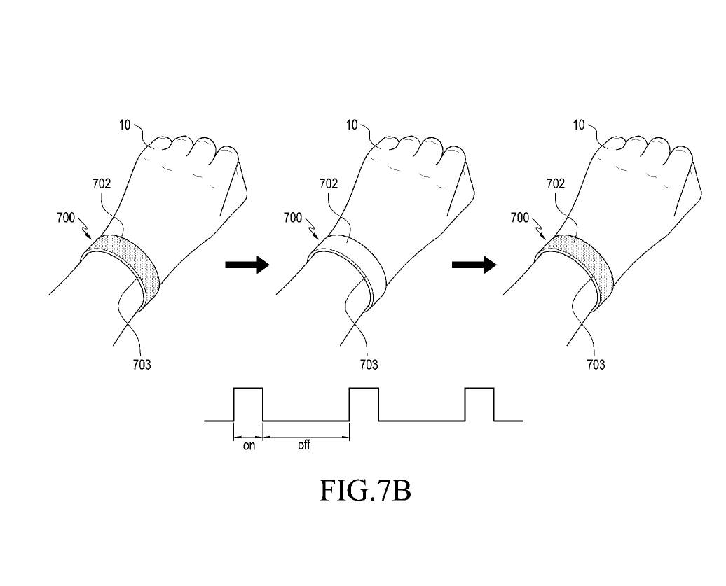

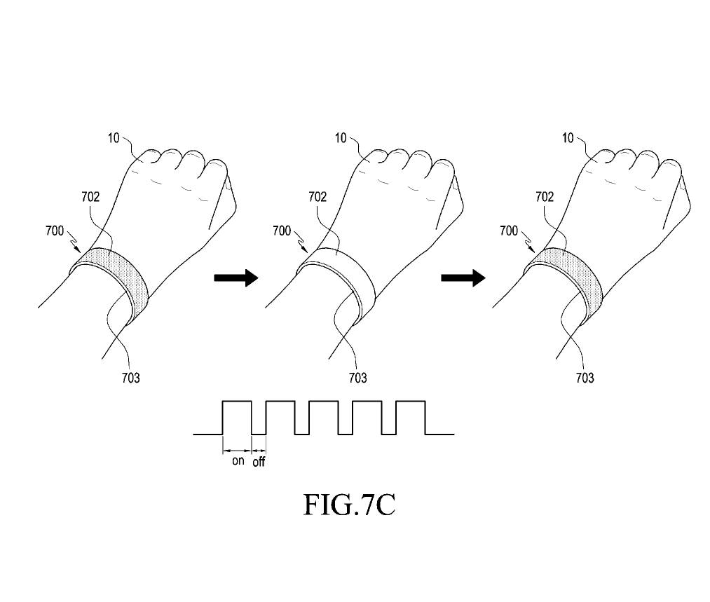

According to various embodiments of the present disclosure, when the pulse rate/pulse cycle of the user 10 is included in a normal range, the processor (for example, the processor 310) may control a first light emitting module 702 or a second light emitting module 703 to continuously output light (or color) as illustrated in FIG. 7A. The term “normal range” may be referred to by various terms, such as a “standard range”, a “normal state”, a “standard state”, or the like. Information on a “reference pulse rate/pulse cycle” for determining whether the pulse rate/pulse cycle of the user 10 is included in the normal range may have been stored in a memory (for example, the memory 360) of a wearable device 700. The term “reference pulse rate/pulse cycle” may be referred to by a term “standard pulse rate/pulse cycle”.

Referring to FIGS. 7B and 7C, when the pulse rate/pulse cycle of the user 10 is changed to be out of the normal range or is decreased or increased compared to the normal range, a processor of the wearable device 700 may control an output cycle of the first light emitting module 702 so as to correspond to the decreased or increased pulse rate/pulse cycle. FIG. 7B illustrates the case where the pulse rate/pulse cycle of the user 10 is out of the normal range and decreased, and FIG. 7C illustrates the case where the pulse rate/pulse cycle of the user 10 is out of the normal range and increased.

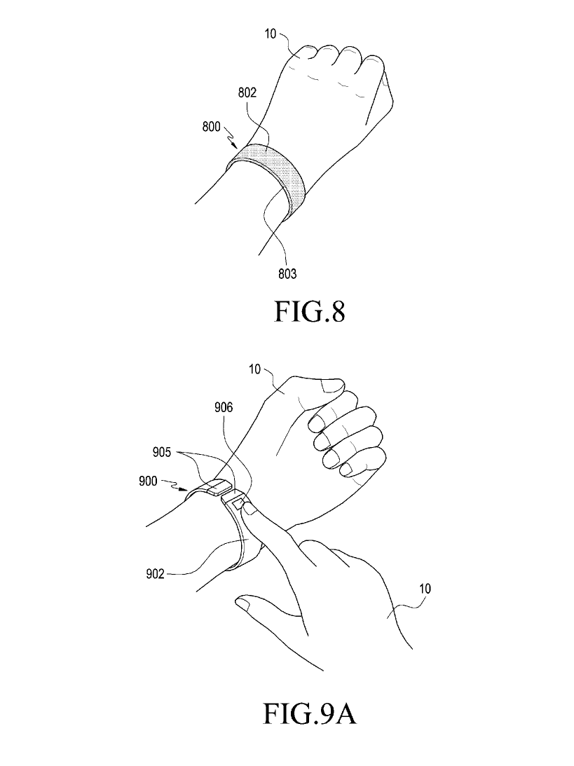

FIGS. 6A to 7C illustrate that the first light emitting module (for example, the first light emitting module 602) might emit light in the first operation mode. According to various embodiments of the present disclosure, with a wearable device 800 in the first operation mode, a second light emitting module 803 and a third light emitting module, as well as a first light emitting module 802, may be controlled to output light for displaying the color as illustrated in FIG. 8.

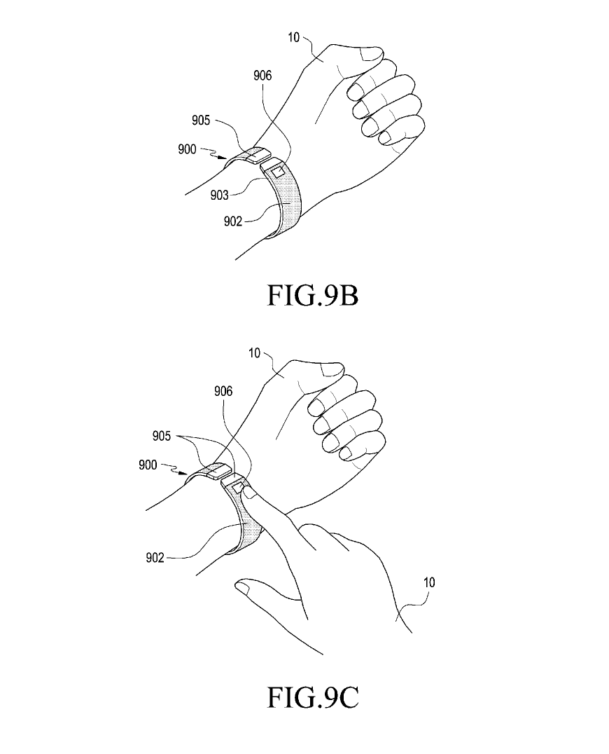

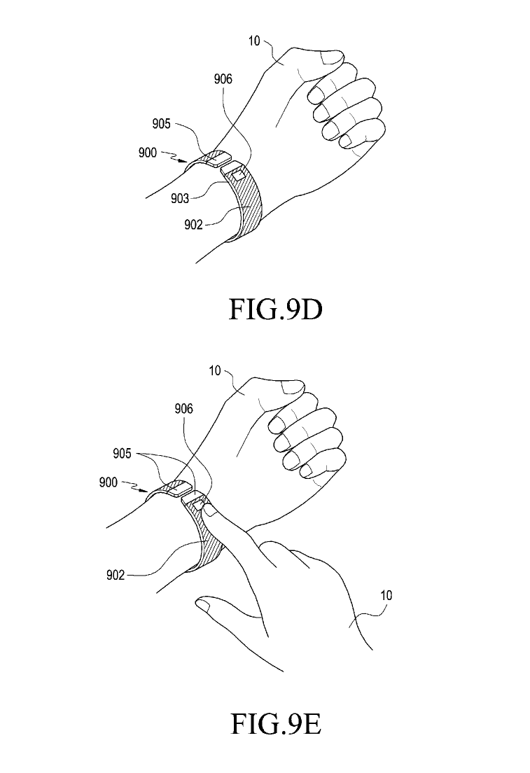

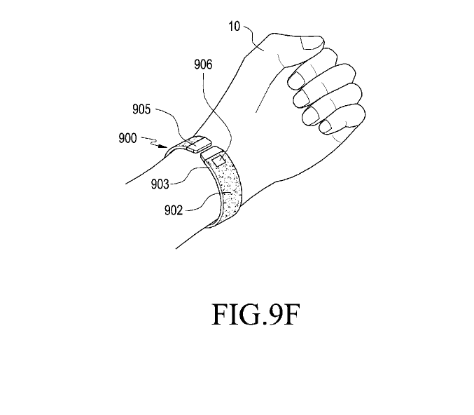

FIGS. 9A to 9F are diagrams for describing a function/operation of changing a color displayed on the wearable device through an input module provided in a wearable device in a first operation mode according to various embodiments of the present disclosure.

Referring to FIGS. 9A to 9F, a wearable device 900 might include a first light emitting module 902, a second light emitting module 903, supporting parts 905, and a button 906. When a button 906 of a wearable device 900 is pressed (for example, the button 906 is pressed once shortly as illustrated in FIG. 4C) during an execution of the first operation mode as illustrated in FIG. 9A, a processor (for example, the processor 310) of the wearable device 900 may control a first light emitting module 902 or a second light emitting module 903 to output a currently set color (for example, green (first color) as illustrated in FIG. 9B. The aforementioned press of the button 906 may mean an entrance to a color change mode.

When the button 906 is re-pressed in a state where the first color is output as illustrated in FIG. 9C, the processor may control the first light emitting module 902 to output a second color (for example, red (second color) different from the first color. When the button 906 is re-pressed by the user 10 in a state where the second color is output as illustrated in FIG. 9E, the processor may control the first light emitting module 902 to output a third color (for example, blue) different from the first color and the second color as illustrated in FIG. 9F. The user 10 may select a color desired to be output through the functions/operations described with reference to FIGS. 9A and 9C. For example, in a case where the user 10 desires to change the color output on the first light emitting module 902 to the second color, when the button 906 is, for example, pressed once for a long time in the state where the second color is output on the first light emitting module 902, the processor may change the first color to the second color and make the second color be output on the first light emitting module 902.

The contents described with reference to FIGS. 9A to 9F are illustratively described based on the performance in the first operation mode, but the aforementioned contents may be equally applied to the second operation mode and/or the third operation mode.

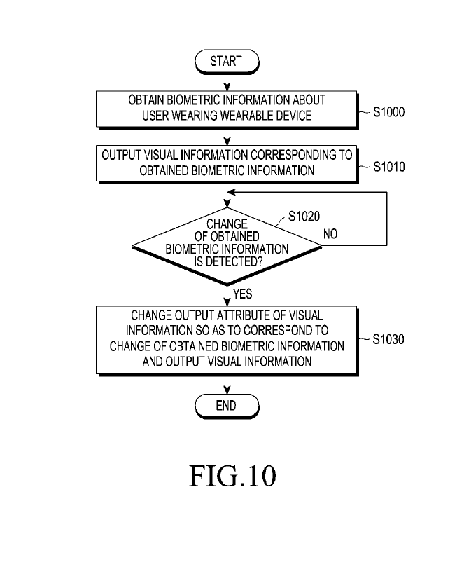

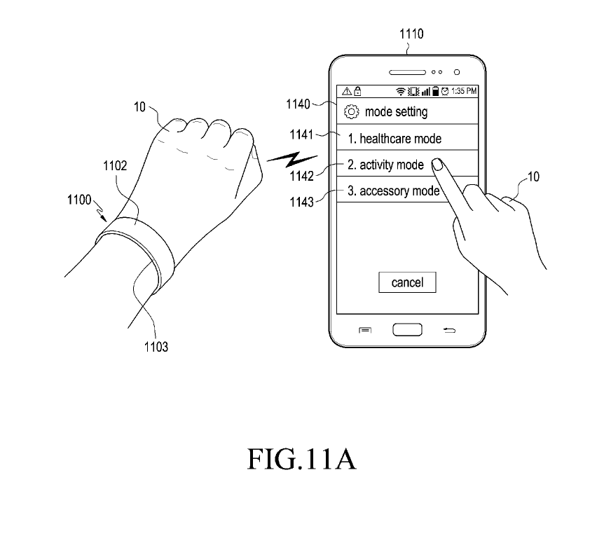

FIG. 10 is a flowchart for describing a method of controlling a wearable device according to various embodiments of the present disclosure.

Referring to FIG. 10, a method of controlling a wearable device according to various embodiments of the present disclosure may include obtaining biometric information about the user 10 wearing the wearable device (for example, the wearable device 300) at operation S1000, outputting visual information corresponding to the obtained biometric information at operation S1010, detecting whether the obtained biometric information is changed while the biometric information is obtained at operation S1020, and when the change of the obtained biometric information is detected, changing an output attribute of the visual information in correspondence to the change of the obtained biometric information and outputting the visual information at operation S1030. In addition, contents which are not described in relation to the method of controlling the wearable device according to various embodiments of the present disclosure may equally refer to the descriptions of the first operation mode of the wearable device (for example, the wearable device 300).

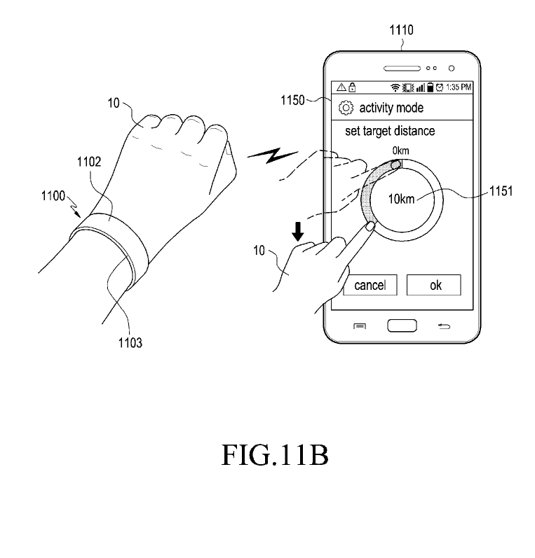

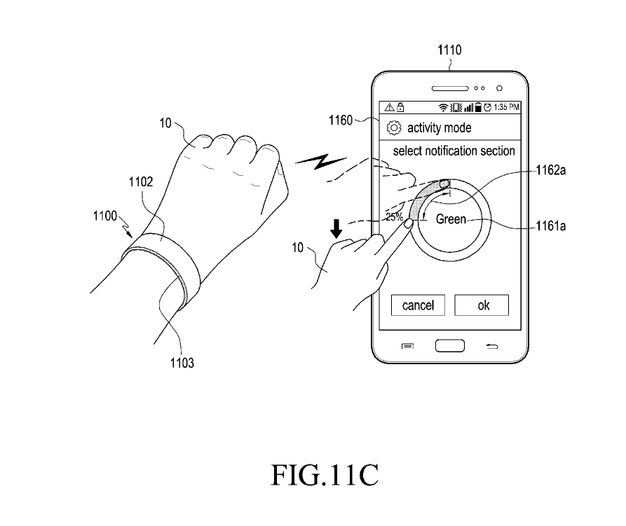

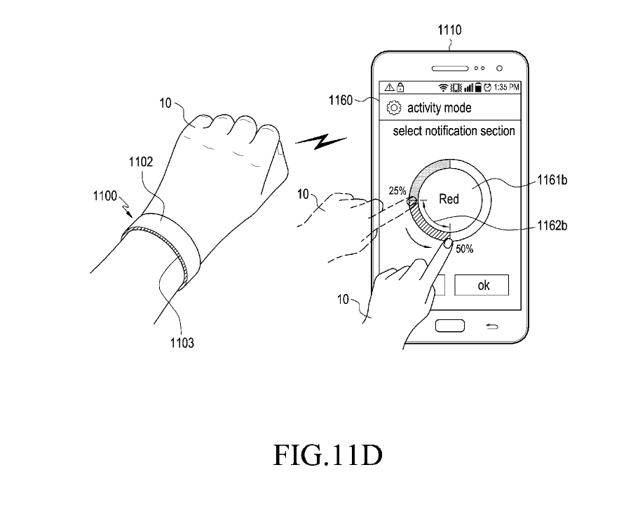

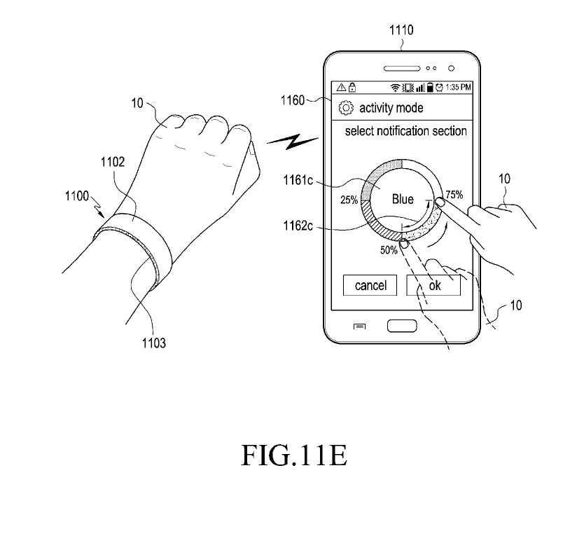

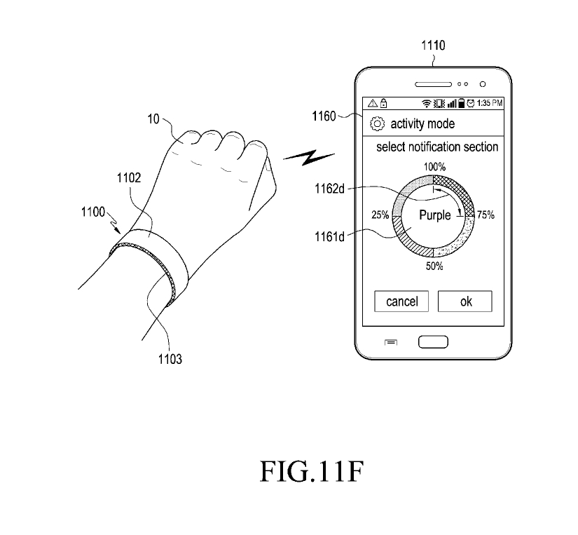

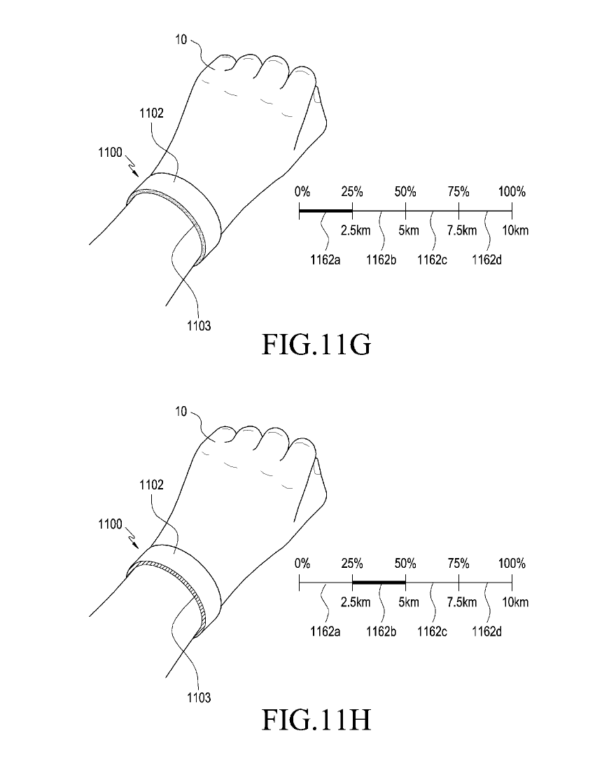

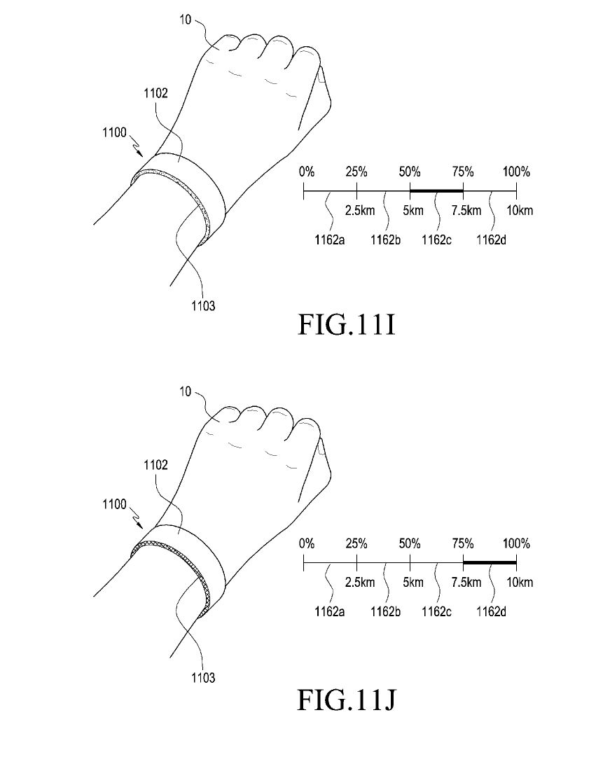

FIGS. 11A to 11J are diagrams for describing various functions/operations performed in a second operation mode of a wearable device according to various embodiments of the present disclosure.

Referring to FIG. 11A, a screen 1140 for setting an operation mode of a wearable device 1100 may include various items 1141, 1142, 1143, and may be displayed on a smart phone 1110. A wearable device 1100 may include a first light emitting module 1102, a second light emitting module 1103, supporting parts 1105, and a button 1106. As illustrated in FIG. 11A, when an execution request for a second operation mode (which may be referred to as an “activity mode” in the present specification) is received from the user 10, a first distance setting screen 1150 may be displayed on the smart phone 1110 as illustrated in FIG. 11B. In the present specification, a “first distance 1151” may be variously referred to as a “target distance”, a “total distance”, and the like. The user 10 may set a target distance, that is a total distance, by which the user 10 desires to jog, through the first distance setting screen 1150 displayed on the smart phone 1110 as illustrated in FIG. 11B. FIG. 11B illustrates the case where the target distance is set to be 10 km.

Referring to FIG. 11C, after the first distance 1151 is set, a notification section setting screen 1160 may be displayed on the smart phone 1110. As illustrated in FIGS. 11C to 11F, the user 10 may set notification section(s) 1162a, 1162b, 1162c, and 1162d through the notification section setting screen 1160 displayed on the smart phone 1110. The notification sections 1162a, 1162b, 1162c, and 1162d mean a distance range set for checking an “achievement” of the user 10 himself/herself, and may be determined based on a current running distance (which may be referred to as a “second distance” in the present specification) from a jogging start point. FIG. 11C illustrates an embodiment in which a section from 0 km to 2.5 km that is 25% of the set first distance 1151 is set as a first notification section 1162a. When the first notification section 1162a is set, a first color (for example, green 1161a) output on a second light emitting module 1103 may be set by a processor of the wearable device 1100 in correspondence to the first notification section 1162a. In order to notify the user 10 of the designation of the first color 1161a, the designated first color 1161a may be output by the second light emitting module 1103 as illustrated in FIG. 11C. The first color 1161a may be changed by the user 10 which will be described below.

FIG. 11D illustrates an embodiment in which a section from 2.5 Km to 5.0 Km that is a range of 25% to 50% of the set first distance 1151 is set as a second notification section 1162b. That is, the second notification section 1162b may include a range larger than 2.5 km and equal to or smaller than 5.0 km. When the second notification section 1162b is set, a second color (for example, red 1161b) output on the second light emitting module 1103 may be designated by a processor of the wearable device 1100 in correspondence to the second notification section 1162b. In order to notify the user 10 of the designation of the second color 1161b, the designated second color 1161b may be output by the second light emitting module 1103 as illustrated in FIG. 11D. The second color 1161b may be changed by the user 10 which will be described below.

FIG. 11E illustrates an embodiment in which a section from 5.0 Km to 7.5 Km that is a range of 50% to 75% of the set first distance 1151 is set as a third notification section 1162c. That is, the third notification section 1162c may include a range larger than 5.0 km and equal to or smaller than 7.5 km. When the third notification section 1162c is set, a third color (for example, blue 1161c) output on the second light emitting module 1103 may be designated by the processor of the wearable device 1100 in correspondence to the third notification section 1162c. In order to notify the user 10 of the designation of the third color 1161c, the designated third color 1161c may be output by the second light emitting module 1103 as illustrated in FIG. 11E. The third color 1161c may be changed by the user 10 which will be described below.

FIG. 11F illustrates an embodiment in which a section from 7.5 Km to 10.0 Km that is a range of 75% to 100% of the set first distance 1151 is set as a fourth notification section 1162d. That is, the fourth notification section 1162d may include a range larger than 7.5 km and equal to or smaller than 10.0 km. When the fourth notification section 1162d is set, a fourth color (for example, purple 1161d) output on the second light emitting module 1103 may be designated by the processor of the wearable device 1100 in correspondence to the fourth notification section 1162d. In order to notify the user 10 of the designation of the fourth color 1161d, the designated fourth color 1161d may be output by the second light emitting module 1103 as illustrated in FIG. 11F. The fourth color 1161d may be changed by the user 10 which will be described below.

Referring to FIGS. 11G to 11J, when the user 10 wearing the wearing device 1100 is located at any one section among the designated first to fourth notification sections 1162a, 1162b, 1162c, and 1162d, any one color among the designated first to fourth colors 1161a, 1161b, 1161c, and 1161d may be output by the second light emitting module 1103. Information on a movement distance of the user 10 may be obtained/detected by, for example, a sensor module (for example, the acceleration sensor 331) of the wearable device 1100. The processor of the wearable device 1100 may calculate the movement distance of the user 10 from a specific point based on the information on the movement distance obtained by the acceleration sensor. According to various embodiments of the present disclosure, the calculation of the movement distance may also be performed by the processor of the smart phone 1110. FIG. 11G illustrates an embodiment, in which the first color 1161a is output by the second light emitting module 1103 at the first notification section 1162a. FIG. 11H illustrates an embodiment, in which the second color 1161b is output by the second light emitting module 1103 at the second notification section 1162b. FIG. 11I illustrates an embodiment, in which the third color 1161c is output by the second light emitting module 1103 at the third notification section 1162c. FIG. 11J illustrates an embodiment, in which the fourth color 1161d is output by the second light emitting module 1103 at the fourth notification section 1162d.

As described above, in the second operation mode, only the second light emitting module 1103 may output various colors according to various embodiments of the present disclosure. It is not necessary to notify neighboring people of an “achievement” for the activity (for example, the jogging) differently from the aforementioned first operation mode and the “achievement” may correspond to personal preference or personal information, so that only the second light emitting module 1103 may output various colors in order to allow only the user 10 of the wearable device 1100 to intuitively recognize the achievement. However, according to various embodiments of the present disclosure, one or more light emitting modes among the first to third light emitting modules 351 to 353 may be controlled so as to output the designated first to fourth colors 1161a, 1161b, 1161c, and 1161d.

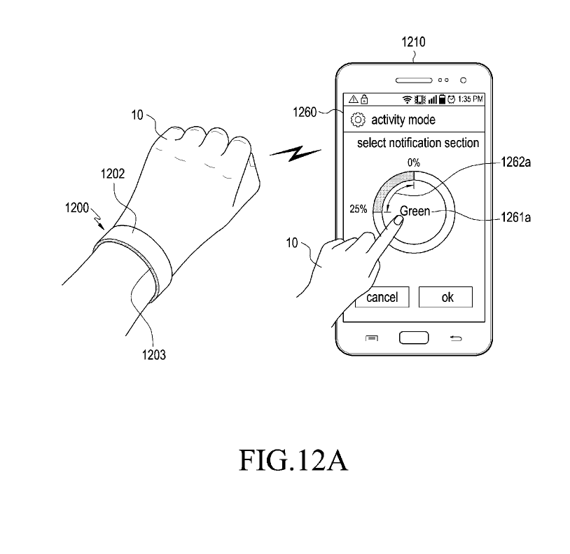

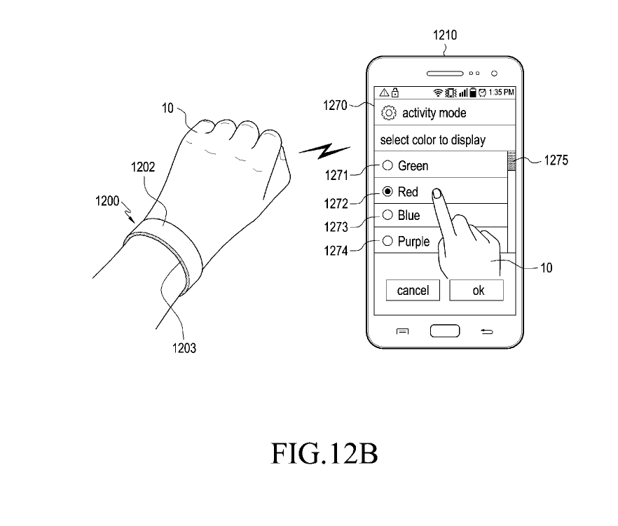

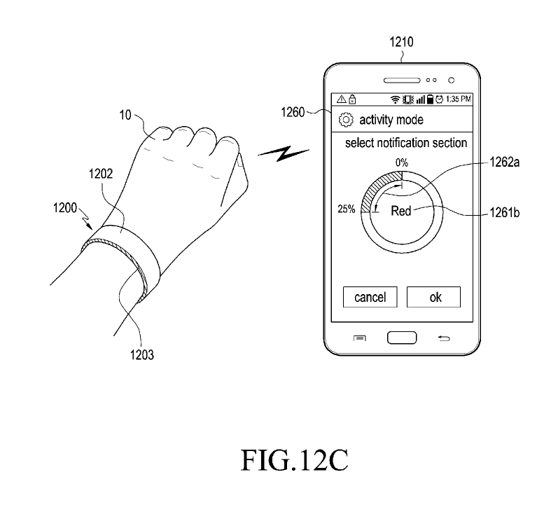

FIGS. 12A to 12C are diagrams for describing a function/operation of changing a color displayed in a wearable device in the second operation mode of the wearable device according to various embodiments of the present disclosure.

Referring to FIG. 12A, a wearable device 1200 may include a first light emitting module 1202, a second light emitting module 1203, supporting parts 1205, and a button 1206. A notification section setting screen 1260 may be displayed on the smart phone 1210. A smart phone 1210 connected with a wearable device 1200 may receive an input for changing the color designated to be output at the notification section (for example, the first notification section 1262a) during the operation of the second operation mode. The wearable device 1200 and the smart phone 1210 may share information about the first notification section and the first color 1261a.

Referring to FIG. 12B, a notification section setting screen 1270 may be displayed on the smart phone 1210. A list of colors 1271, 1272, 1273, and 1274 output table by the wearable device 1200 may be displayed on the smart phone 1210 connected with the wearable device 1200 in order to change the designated color. The user 10 may additionally search the list of the output table colors by controlling a scroll bar 1275. The user 10 may select any one color (for example, red 1272) in the list of the output table colors 1271, 1272, 1273, and 1274 as illustrated in FIG. 12B.

Referring to FIG. 12C, the wearable device 1200 may receive information about a color (for example, red 1272) changed by the user 10, and a processor (for example, the processor 310) of the wearable device 1200 may control a second light emitting module 1203 so as to output the changed color 1272 as a second notification color 1261b.

In FIGS. 12A to 12C, the embodiment, in which the designated color is changed through the smart phone 1210 connected with the wearable device 1200, has been described, but as described above, the designated color may also be controlled to be changed through an input module (for example, the input module 340) of the wearable device 1200.

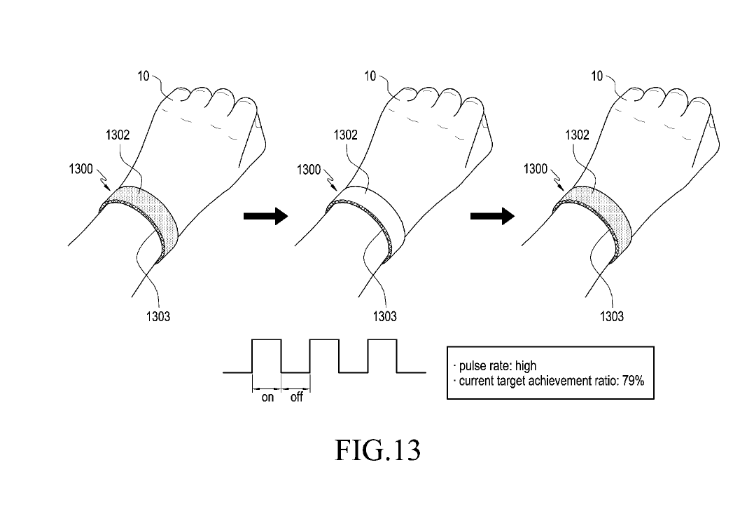

FIG. 13 is a diagram for describing a function/operation simultaneously performed in various operation modes of a wearable device according to various embodiments of the present disclosure.

According to various embodiments, as illustrated in FIG. 13, the first operation mode and the second operation mode may be simultaneously applied. A wearable device 1300 may include a first light emitting module 1302, a second light emitting module 1303, supporting parts 1305, and a button 1306. For example, it is possible to obtain biometric information (for example, a pulse rate/pulse cycle) of the user during an activity (for example, jogging) of the user, and simultaneously provide visual information about an “achievement” and visual information about the “biometric information” through a first light emitting module 1302 and a second light emitting module 1303. FIG. 13 illustrates an example of a function/operation of outputting visual information in a case where an achievement for the activity of the user 10 is 79%, and the pulse rate/pulse cycle is increased.

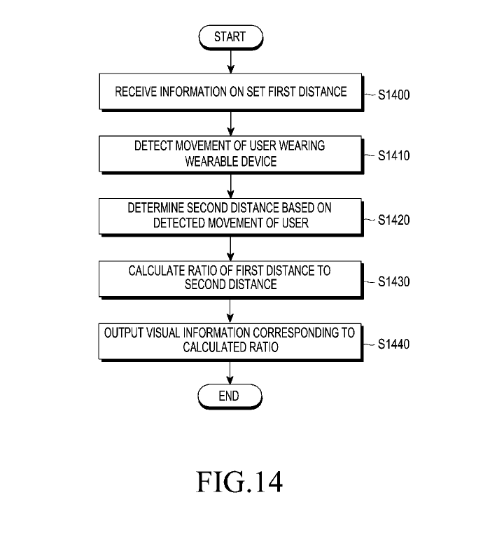

FIG. 14 is a flowchart for describing a method of controlling a wearable device according to various embodiments of the present disclosure.

Referring to FIG. 14, a method of controlling a wearable device (for example, the wearable device 300) according to various embodiments of the present disclosure may include an operation S1400 of receiving information on a first distance set by a user and an operation S1410 of detecting a movement of the user 10 wearing the wearable device. The method may include an operation S1420 of determining a second distance based on the detected movement of the user 10 after the detecting of the movement of the user, and an operation S1430 of calculating a ratio of the first distance to the second distance. The method may include an operation S1440 of outputting visual information corresponding to the calculated ratio. However, the aforementioned operations S1420 and S1430 may also be performed by an external electronic device (for example, the smart phone) electrically connected with the wearable device. In this case, the wearable device may perform the operation S1440 after receiving the information on the second distance calculated by the external electronic device and the information on the ratio of the first distance to the second distance. The description of the second operation mode may be equally applied to the method of controlling the wearable device according to various embodiments of the present disclosure described with reference to FIG. 14.

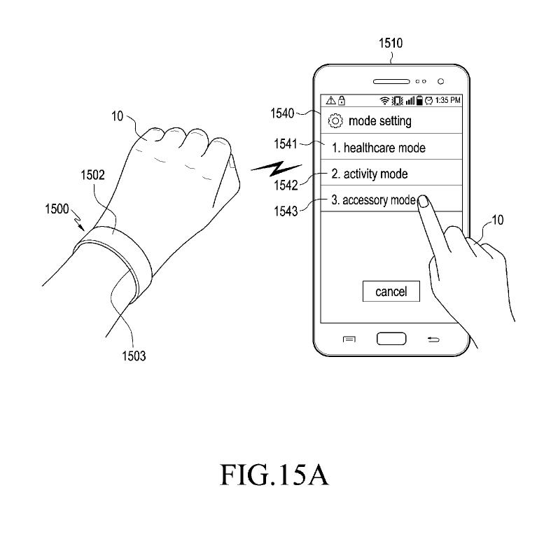

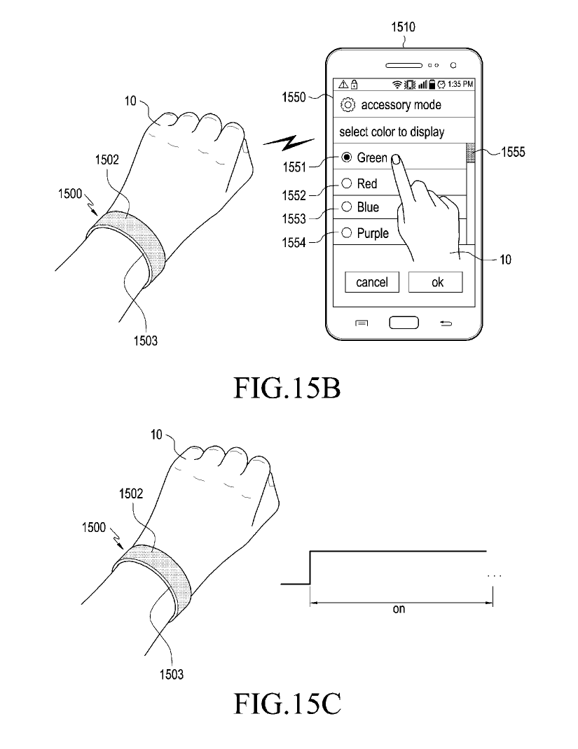

FIGS. 15A to 15C are diagrams for describing various functions/operations performed in the third operation mode of the wearable device according to various embodiments of the present disclosure.

Referring to FIG. 15A, a wearable device 1500 may include a first light emitting module 1502, a second light emitting module 1503, supporting parts 1505, and a button 1506. A screen 1540 for setting an operation mode of a wearable device 1500 may include various items 1541, 1542, and 1543, and may be displayed on an external electronic device (for example, a smart phone 1510). As illustrated in FIG. 15A, when an execution request for a third operation mode (which may be referred to as an “accessory mode” in the present specification) is received from the user 10, a color selection screen 1550 may be displayed in order to execute the third operation mode as illustrated in FIG. 15B. A list of colors 1551, 1552, 1553, and 1554 outputable on the wearable device 1500 may be displayed on the color selection screen 1550. When any one color is selected in the list of the colors 1551, 1552, 1553, and 1554 from the user 10, the wearable device 1500 may receive information on the selected color (for example, green 1551) from the smart phone 1510. Additional colors may be viewed and selected by manipulation of a scroll bar 1555. A processor (for example, the processor 310) of the wearable device 1500 may control the first light emitting module 1502, so that the selected color 1551 is continuously output based on the received information. An accessory effect may be exhibited by the function(s) or the operation(s) performed in the third operation mode.

The term “unit” or “module” as used in various embodiments of the present disclosure may, for example, include one of hardware, software, and firmware, or a combination of two or more thereof. The “unit” or “module” may be used interchangeably with, for example, the term “logic”, “logical block”, “component”, or “circuit”. The “unit” or “module” may be mechanically or electronically implemented. For example, the “module” according to various embodiments of the present disclosure may include at least one of an application-specific integrated circuit (ASIC) chip, a field-programmable gate arrays (FPGAs), and a programmable-logic device for performing operations which have been known or are to be developed hereafter.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. A wearable device comprising:
a processor;
a sensor module configured to obtain biometric information about a user wearing the wearable device; and
a light emitting module configured to output visual information corresponding to the obtained biometric information,
wherein the processor controls an output attribute of the visual information to be changed and output according to a change in the obtained biometric information.

2. The wearable device of claim 1, wherein the biometric information includes information on a pulse cycle of the user, and
the visual information includes at least one color output by the light emitting module.

3. The wearable device of claim 2, wherein the output attribute includes an output period of a color output by the light emitting module or a kind of color output by the light emitting module.

4. The wearable device of claim 3, wherein when the pulse cycle is decreased, the processor controls the output period to be decreased and output according to the decreased pulse cycle.

5. The wearable device of claim 3, wherein when the pulse cycle is increased, the processor controls the output period to be increased and output according to the increased pulse cycle.

6. The wearable device of claim 3, wherein when the pulse cycle is increased or decreased, the processor controls a color output by the light emitting module to be output according to a predetermined output period.

7. A method of controlling a wearable device, the method comprising:
obtaining biometric information about a user wearing the wearable device;
outputting visual information corresponding to the obtained biometric information; and
changing an output attribute of the visual information according to a change of the obtained biometric information and outputting the visual information.

8. The method of claim 7, wherein the biometric information includes information on a pulse cycle of the user, and
wherein the visual information includes at least one color output by the light emitting module.

9. The method of claim 8, wherein the output attribute includes an output period of a color output by the wearable device or a kind of color output by the wearable device.

10. The method of claim 9, further comprising:
when the pulse cycle is decreased, decreasing the output period according to the decreased pulse cycle and outputting the visual information.

11. The method of claim 9, further comprising:
when the pulse cycle is increased, increasing the output period according to the decreased pulse cycle and outputting the visual information.

12. The method of claim 9, further comprising:
when the pulse cycle is increased or decreased, outputting a color output by the wearable device according to a predetermined output period.

13. A wearable device comprising:
a light emitting module;
a sensor module configured to detect a movement of a user wearing the wearing device;
a communication module configured to receive information on a first distance set by the user; and
a processor configured to calculate a ratio of the received first distance to a second distance determined based on a movement of the user,
wherein the processor controls visual information corresponding to the calculated ratio to be output by the light emitting module.

14. The wearable device of claim 13, wherein the light emitting module includes a first light emitting module, a second light emitting module, and a third light emitting module.

15. The wearable device of claim 14, wherein the processor controls visual information corresponding to the ratio to be output by the second light emitting module.

16. The wearable device of claim 14, wherein the processor controls visual information corresponding to the ratio to be output by the second light emitting module and the third light emitting module.

17. The wearable device of claim 13, wherein the visual information includes a color output in correspondence to the calculated ratio.
